(19) United States
(12) Patent Application Publication
(10) Pub. No.: US 2010/0113929 A1
(43) Pub. Date: May 6, 2010

(54) High-frequency ultrasonic imaging system and method

(75) Inventors: Chih Kuang Yeh, Zhongli City (TW); Chih Hao Cheng, Hsnichu (TW); Chao Hung Chung, Hsinchu (TW)

Correspondence Address: LOWE HAUPTMAN HAM & BERNER, LLP, 1700 DIAGONAL ROAD, SUITE 300, ALEXANDRIA, VA, 22314, US

(73) Assignee: NATIONAL TSING HUA UNIVERSITY, Hsinchu (TW)

(21) Appl. No.: 12/289,634

(22) Filed: Oct. 31, 2008

(51) Int. Cl. A61B 8/14

(52) U.S. Cl. 600/443

(57) ABSTRACT

Since high-frequency ultrasound provides the advantage of high spatial resolution, it has been applied to relevant fields of medical imaging research. Due to a lack of high-frequency ultrasonic transducer with array structure, the high-frequency ultrasonic transducer must be performed in a fixed depth of focus during scanning. A swept scanning method is typical for flow estimation. However, this method cannot provide the precise flow estimation within the irregular-shaped object, because the focal zone of the above-mentioned transducer just covers a specific depth of focus and the outside the focal area corresponds poor signal to noise ratio. To resolve this problem, the present invention provides a skin scanning method, which can move the transducer along a scanning route parallel to the contour of the irregular-shaped object during scanning. The scanning results show that the skin scanning method improves the accuracy of flow estimation.

### TECHNICAL FIELD

The invention is related to a high-frequency ultrasonic imaging system and method, and more particularly to an ultrasonic transducer moved along a scanning route parallel to the contour of an object during the scanning steps of a skin scanning method.

### BACKGROUND

Nowadays, the commercial ultrasonic equipments used widely in hospitals are related to low-frequency ultrasound (less than 5 MHz), and thus their resolution is relatively low. The resolution of the ultrasonic system is in inverse proportion to its frequency. In order to have better resolution, the ultrasonic system has to use high-frequency ultrasound higher than 15 MHz. Since high-frequency (e.g. 25 MHz) ultrasound has the advantage of high spatial resolution, it has been applied to relevant fields of medical imaging research. However, since the transducer of the high-frequency ultrasonic equipment is quite expensive, and lacks array structures, it usually utilizes the swept scanning method to obtain the Doppler image for a two-dimension (2-D) blood flow information. Typically, the high-frequency ultrasonic image for estimating the blood flow on a tumor 14 located in an animal body 8 is based on a swept scanning method recently, shown as FIG. 1. According to the swept scanning method for conventional high-frequency ultrasonic imaging system, the pulser/receiver utilizes the ultrasonic transducer 10 to transmit ultrasonic pulses and receive echoes while the ultrasonic transducer is slightly moved horizontally along a scanning route 12 and is driven by a servo-motor (not shown) so as to perform the ultrasonic image in real time.

However, the ultrasonic transducer 10 is horizontally moved in the Y axis during the scanning steps of swept scanning method. Thus, the ultrasonic transducer 10 only has a fixed depth D of focus (i.e. a specific focal depth D) for estimating the blood flow in the angiogenesis 16 on a surface of the tumor 14. A focal zone 22 of the ultrasonic transducer 10 is formed by the specific focal depth D, and is extended horizontally. The focal zone 22 has a high spatial resolution, a clear image and the precise blood flow estimation. The outside of the focal zone 22 (i.e. non-focal zone 24) has the lower spatial resolution and the lower ratio of signal to noise such that a weaker signal of blood flow cannot be analyzed and distinguished from the noise. It is interesting to the researchers that the growth of tumor 14 depends on that of the angiogenesis 16 located thereon. Since the angiogenesis 16 grow up on the surface of the tumor 14 which is irregular, the focal zone 22 only covers a part of angiogenesis 16 at two intersections 18 of the angiogenesis 16 and the focal zone 22, and the non-focal zone 24 covers the other part of angiogenesis 16. Thus, the ultrasonic transducer 10 which has the specific focal depth D cannot effectively estimate the micro-circulation flow in the angiogenesis 16 located on the surface of the tumor 14.

Accordingly, there exists a need for a high-frequency ultrasonic imaging system and method capable of solving the above-mentioned problems.

### SUMMARY

the present invention to provide an ultrasonic imaging system and method, including an ultrasonic transducer which is moved along a scanning route parallel to a contour of the first object, whereby a focal zone of the ultrasonic transducer formed by the specific focal depth is extended along the contour of the first object, and the focal zone of the ultrasonic transducer has the precise flow estimation capable of solving the above-mentioned problems in the prior art.

The present invention provides an ultrasonic imaging method (named as a skin scanning method) for estimating a surface of the first object (e.g. tumor) located in the second object (e.g. animal body). The ultrasonic imaging method includes the following steps of: acquiring a contour of the first object; moving an ultrasonic transducer along a scanning route parallel to the contour of the first object for transmitting high-frequency ultrasonic pulses and receiving echoes; forming a focal zone by a specific focal depth of the ultrasonic transducer during moving, wherein the focal zone is extended along the contour of the object for estimating the surface of the first object; processing the received echoes resulted from the focal zone so as to form a preliminary image; reconstructing the preliminary image to a final image; and displaying the final image. The skin scanning method further includes the following step of: calculating a blood flow velocity in the third object (e.g. angiogenesis) on the surface of the first object.

The present invention further provides an ultrasonic imaging system for estimating a surface of the first object located in the second object. The ultrasonic imaging system includes an ultrasonic transducer, a driving device and an electronic device. The ultrasonic transducer transmits high-frequency ultrasonic pulses and receives echoes. The driving device is physically connected to the ultrasonic transducer for driving the ultrasonic transducer to move along a scanning route parallel to the contour of the first object. The electronic device is electrically connected to the driving device and the ultrasonic transducer for controlling the driving device, acquiring the contour of the first object, processing the received echoes, reconstructing an image and displaying the image.

The ultrasonic imaging system and method include the ultrasonic transducer which is adapted to estimate the blood flow in the angiogenesis on the surface of the tumor. Since the ultrasonic transducer of the present invention is moved along a scanning route parallel to the contour of the tumor, the focal zone of the ultrasonic transducer formed by the specific focal depth is extended along the contour of the tumor. The focal zone of the ultrasonic transducer has the precise blood flow estimation. It is interesting that the growth of tumor depends on that of angiogenesis located on the tumor. Although the angiogenesis grow up on the surface of the tumor which is irregular, the focal zone still covers the whole angiogenesis, because the focal zone of the ultrasonic transducer is extended along the contour of the tumor. Thus, the ultrasonic transducer which has the specific focal depth can effectively estimate the micro-circulation flow in the angiogenesis located on the surface of the tumor.

The foregoing, as well as additional objects, features and advantages of the invention will be more apparent from the following detailed description, which proceeds with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. Embodiments of the present invention are illustrated by way of example, and not by limitation, in the figures of the accompanying drawings, wherein elements having the same reference numeral designations represent like elements throughout and wherein:

FIG. 1 is a cross-sectional view of a swept scanning method in the prior art.

FIG. 2 is a block schematic diagram of a high-frequency ultrasonic imaging system according to an embodiment of the present invention.

FIG. 3 is a cross-sectional view of a skin scanning method of the present invention.

FIG. 4 is a flow diagram of a high-frequency ultrasonic imaging method of the present invention.

FIG. 5 shows that the real route of the moved servo-motor is slightly different the ideal route of the moved servo-motor.

FIG. 6 is an image shows the contour of the object.

FIG. 7 is an image shows that all boundary positions form the contour of the object.

FIG. 8 shows the malposition of the original shape of the object on the preliminary image.

FIG. 9 is two images show the preliminary image is reconstructed to a final image.

FIG. 10 is a color Doppler image of a flow phantom showing the scanning result during the skin scanning method and the swept scanning method.

FIG. 11 shows the distribution of the blood flow velocities in the three blood vessels during the skin scanning method and the swept scanning method.

FIG. 12 shows the distribution of the blood flow velocities in the cross-sections of the three blood vessels during the skin scanning method and the swept scanning method.

FIG. 13 shows that the flow estimation in the three blood vessels in six experiments during the skin scanning method and the swept scanning method.

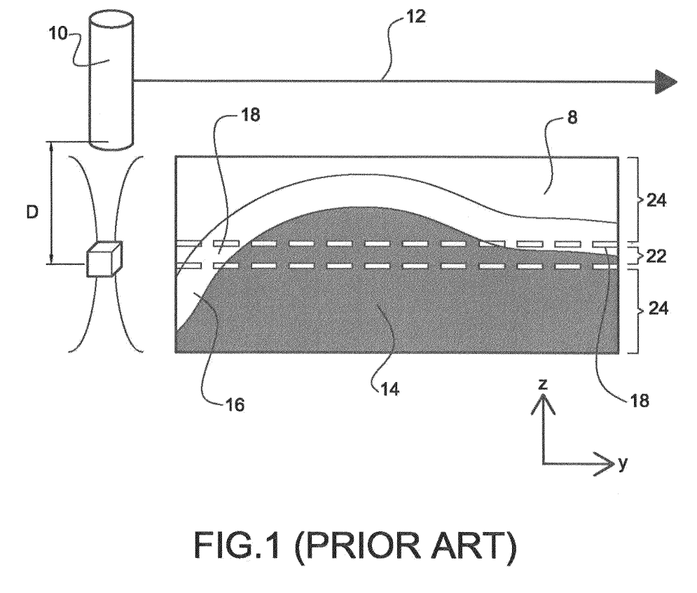

### DETAILED DESCRIPTION OF THE PREFERRED EMBODIMENT

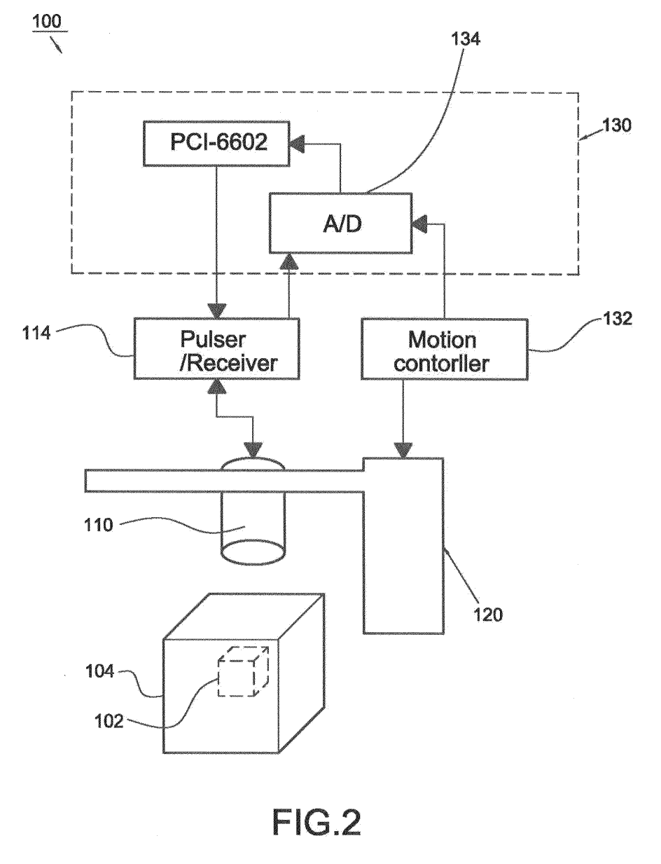

Referring to FIG. 2, it depicts a high-frequency ultrasonic imaging system according to an embodiment of the present invention. The high-frequency ultrasonic imaging system 100 includes an ultrasonic transducer 110, a pulser/receiver 114, a driving device 120 and an electronic device 130. The ultrasonic transducer 110 is made of piezoelectricity material and has the structure of single element. The pulser/receiver 114 utilizes the ultrasonic transducer 110 to transmit high-frequency ultrasonic pulses and receive echoes. A motion controller 132 can control the driving device 120. The driving device 120 is physically connected to the ultrasonic transducer 110 for driving the ultrasonic transducer 110 to simultaneously move horizontally and vertically. More detailed, the driving device 120 can be a servo-motor mechanism (not shown) for driving the ultrasonic transducer to simultaneously move horizontally and vertically, whereby the ultrasonic transducer 110 can be moved along a scanning route 112 parallel to the contour of the first object 102. The first object 102 is located in the second object 104. The electronic device 130, e.g. personal computer (PC) which includes a processor (not shown) and a monitor (not shown), is electrically connected to the driving device 120 and the ultrasonic transducer 110 through the motion controller 132 and the pulser/receiver 114 respectively for controlling the driving device 120, acquiring the contour of the first object 102, processing the received echoes, reconstructing the image, calculating a flow velocity and displaying the image etc. The received echoes (RF signals) are amplified by an amplifier (not shown), and are converted to digital data by an analogy-to-digital converter (A/D) 134. The digital data are analyzed by the electronic device 130, and then are stored in a memory of the electronic device 130.

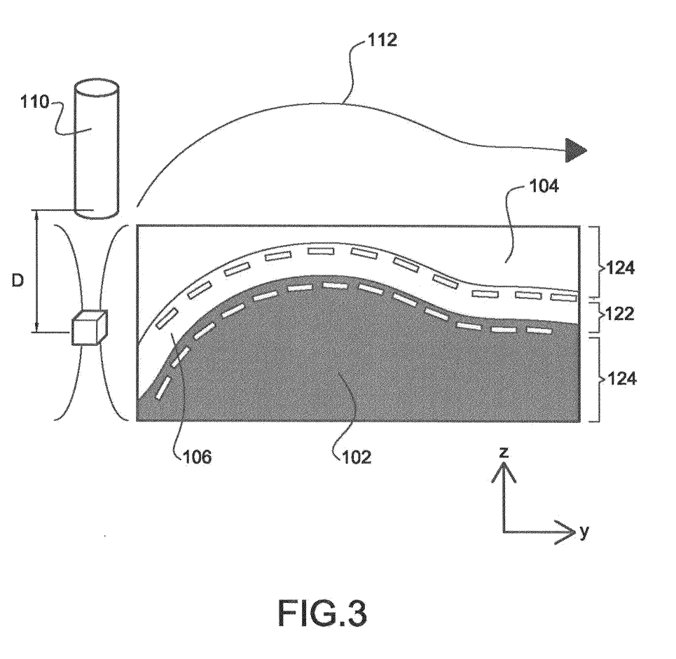

Referring to FIG. 3, the first object 102 is located in the second object 104. The ultrasonic transducer 110 can be moved along the scanning route 112 parallel to the contour of the first object 102 during scanning, because the ultrasonic transducer 110 can be simultaneously moved horizontally in the Y axis and vertically in Z axis by the driving device 120. These scanning and imaging steps are named as a “skin scanning method”. According to the skin scanning method for the high-frequency ultrasonic imaging system 100 of the present invention, the ultrasonic transducer 110 transmits ultrasonic pulses and receives echoes during the movement of the ultrasonic transducer 110 so as to perform the ultrasonic image in real time. In particular, the ultrasonic transducer 110 also has a fixed depth D of focus (i.e. a specific focal depth D) along the scanning route 112 for estimating a surface of the object 102, but a focal zone 122 of the ultrasonic transducer 110 formed by the specific focal depth D is extended along the contour of the first object 102. The focal zone 122 has a high spatial resolution, a clear image and the surface estimation. The outside of the focal zone 122 (i.e. non-focal zone 124) has the lower spatial resolution and the lower ratio of signal to noise.

In this embodiment, the first object 102 can be a tumor, and the second object 104 can be a human body or an animal body. The ultrasonic transducer 110 is adapted to estimate the blood flow in angiogenesis (i.e. the third body 106) on a surface of the tumor by utilizing the skin scanning method of the present invention. The focal zone 122 of the ultrasonic transducer 110 formed by the specific focal depth D is extended along the contour of the tumor. The focal zone 122 has the precise blood flow estimation. It is interesting that the growth of tumor depends on that of the angiogenesis located on the surface of the tumor. Although the angiogenesis grow up on the surface of the tumor which is irregular, the focal zone 122 still covers the whole angiogenesis, because the focal zone 122 of the ultrasonic transducer 110 is extended along the contour of the tumor. Thus, the ultrasonic transducer 110 which has the specific focal depth D can effectively estimate the micro-circulation flow in the angiogenesis located on the surface of the tumor.

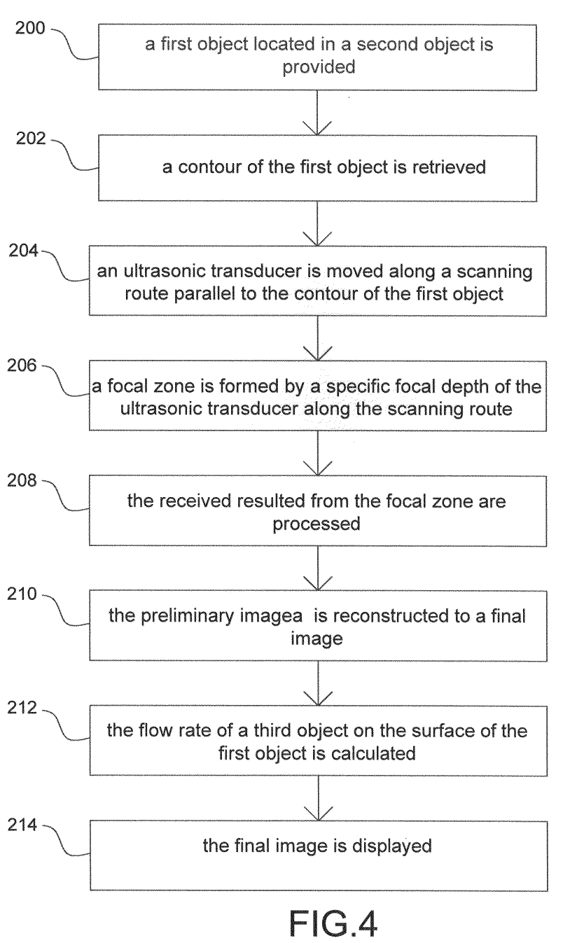

Referring to FIG. 4, it depicts a high-frequency ultrasonic imaging method using the high-frequency ultrasonic imaging system 100 of the present invention. The high-frequency ultrasonic imaging method is also named as a skin scanning method. The skin scanning method includes steps as follows. In the step 200, a first object 102 (e.g. tumor) located in a second object 104 (e.g. human body or animal body) is provided, shown in FIG. 3. In the step 202, a contour of the first object 102 is retrieved. In the step 204, an ultrasonic transducer 110 is moved along a scanning route 112 parallel to the contour of the first object 102 for transmitting high-frequency ultrasonic pulses and receiving echoes. In the step 206, a focal zone 122 is formed by a specific focal depth D of the ultrasonic transducer 110 during moving, wherein the focal zone 122 is extended along the contour of the first object 102 for estimating a surface on the first object 102. In the step 208, the received echoes resulted from the focal zone 122 are processed so as to form a preliminary image. In the step 210, the preliminary image is reconstructed to a final image. In the step 212, the flow velocity in a third object 106 (e.g. angiogenesis) on the surface of the first object 102 is calculated. In the step 214, the final image is displayed.

In order to realize the “skin scanning method” of the present invention, more detailed descriptions of the steps of the high-frequency ultrasonic imaging method are as follows.

The construction of a driving device:

The driving device can be a servo-motor mechanism for driving the ultrasonic transducer. The servo-motor mechanism is a piezoelectricity-ceramic motor (HR8, Galil Motion Control, Rocklin, Calif.) includes a motor driver (AB1A, Galil Motion Control, Rocklin, Calif.) and a motor controller (Model DMC-2040, Galil Motion Control, Rocklin, Calif.). The motor controller calculates and processes the control program of the motor motion, and sends the control code of the servo-motor motion to the motor driver via an isolating terminal bus (ICM-2900, Galil Motion Control, Rocklin, Calif.) so as to drive the servo-motor mechanism. The maximum output force of the servo-motor mechanism is 32 NT. The minimum scale of an optical Meter is 0.1 μm. Since the skin scanning method can be based on the conventional swept scanning method, the servo-motor must be simultaneously moved vertically in Z axis when the servo-motor is moved horizontally in the Y axis, whereby the servo-motor can moved along a scanning route of any shape. Thus, the control program of the servo-motor has a further function, such that the motions of the servo-motor mechanism in the Y-axis and Z-axis are independent, the servo-motor mechanism is moved vertically to a predetermined position in the Z-axis when the servo-motor is moved horizontally to a predetermined distance in the Y-axis. The operation interface of the high-frequency ultrasonic imaging system of the present invention can be a personal computer (PC), and control programs of all components can be written by C++ program. Thus, the necessary position sequence of the servo-motor mechanism in the Z-axis can be also inputted by the personal computer, and then the motor controller sends the control code of the servo-motor motion to a memory of the motor driver.

After the contour of the object is retrieved, it is hopeful that the ultrasonic transducer can be moved along the scanning route parallel to the contour of the object by the servo-motor mechanism. If the servo-motor mechanism would be moved vertically to an infinite distance in the Z-axis while the servo-motor mechanism is moved horizontally to a unit of distance in the Y-axis, the servo-motor mechanism can be moved along an ideal route parallel to the contour of the object. However, the maximum output forces of the servo-motor mechanism in the Y-axis and the Z-axis are limited (e.g. 32 NT). In other words, the servo-motor mechanism is moved vertically to a limited distance in the Z-axis while the servo-motor mechanism is moved horizontally to a unit of distance in the Y-axis. According to the maximum output forces of the servo-motor mechanism and the loading thereof, the maximum acceleration of the servo-motor mechanism should be 6.71 m/sec². The servo-motor mechanism is moved to a maximum speed in the Z-axis during a pulse each time, the speed value is shown as:

V_{z}=aₘₐₓ/PRF

aₘₐₓ is the maximum acceleration of the servo-motor mechanism, PRF indicates pulse repetition frequency from the ultrasonic transducer. Since the servo-motor mechanism must be moved horizontally and uniform, the horizontal speed value of the servo-motor mechanism in the Y-axis is shown as:

V_{Y}=PRF*dy

dy is the horizontal distance of emitting each pulse from the ultrasonic transducer. According to the above-mentioned two equations, the servo-motor mechanism is moved vertically to the following maximum distance in the Z-axis while the servo-motor mechanism is moved horizontally to a unit of distance in the Y-axis:

$$\frac{{a}_{max} / PRF ( 1 / s )}{PRF ( 1 / s ) \times dy ( 0.1 um )} = \frac{{a}_{max}}{{PRF}^{2} \times dy} = \frac{67107840}{{PRF}^{2} \times dy}$$

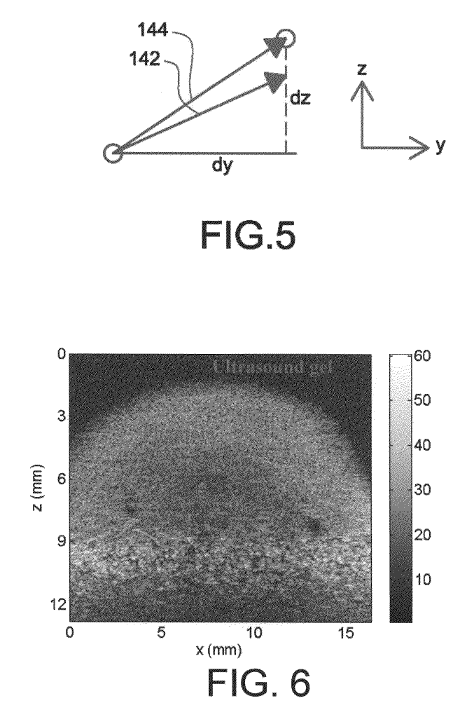

For example, when PRF=512 (1/sec) and dy=10 (0.1 um), the maximum distance of the servo-motor mechanism in the Z-axis is 26; and when PRF=1024 (1/sec) and dy=10 (0.1 um), the maximum distance of the servo-motor mechanism in the Z-axis is 6. In order to protect the servo-motor mechanism, the output force of the servo-motor mechanism is generally less than the maximum output force. Thus, the real route 142 of the moved servo-motor mechanism can be slightly different the ideal route 144 which is parallel to the contour of the object, shown in FIG. 5.

The acquisition of the contour of the object:

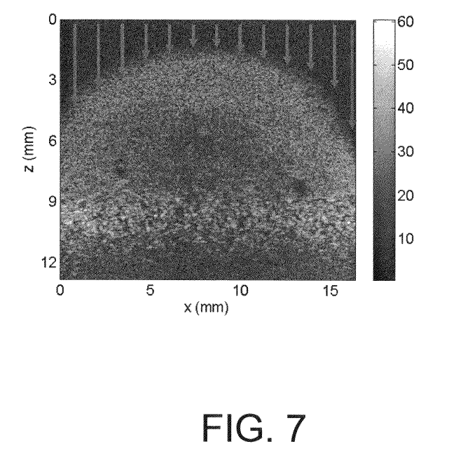

In order to move the ultrasonic transducer along a scanning route parallel to the contour of the object, the uneven condition in the surface of the scanned object must be quantified to scales and then inputted to the driving device. A prior scan for the object is executed by brightness mode (B-mode) scanning steps. After the image is acquired, the contour of the object is calculated by the program immediately. In order to avoid the scatter of ultrasound through the air during the brightness scanning steps before the ultrasound is transmitted to the object, water or ultrasound coupling gel can be acted as a medium located between the ultrasonic transducer and the object for matching the acoustic impedance therebetween. Since the ultrasound is reflected quite weakly in the water or ultrasound coupling gel, the brightness of it on the image is lower. But, the ultrasound is reflected relatively strongly in a body of the object, the brightness of it on the image is higher, whereby the contrast of the contour of the object is displayed evidently, shown in FIG. 6. According to the property of the brightness of the image, the brightness of the boundary of the object can be set to a threshold by an experiment. The program searches from the upper part to the lower part of the image. When the brightness of the position of the image is more than the threshold, the boundary position is recorded. All boundary positions form the contour of the object, shown in FIG. 7.

The reconstruction of the preliminary image to a final image:

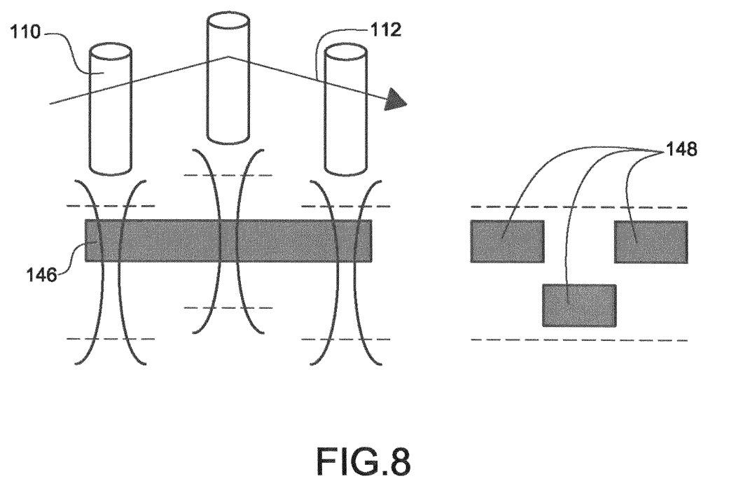

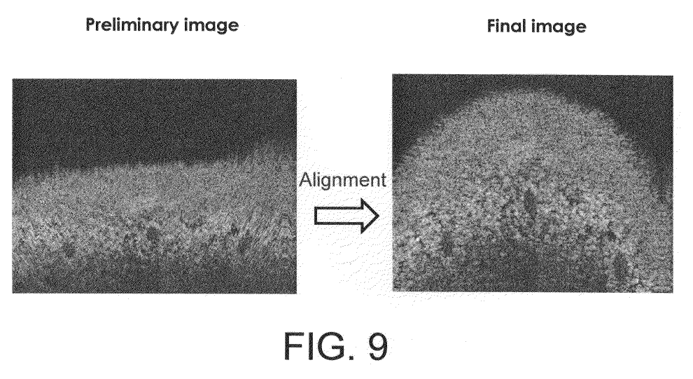

Since the ultrasonic transducer 110 is moved along the scanning route 112 parallel to the contour of the object, the ultrasonic transducer 110 can be moved upward and downward. Thus, the original shape 146 of the object on the preliminary image can has a malposition. The original shape 146 in the same horizontal level may be distributed to the different positions on the preliminary image such as 148, on the other hand the original shape in the different horizontal level may be distributed to the same positions on the preliminary image, shown in FIG. 8. Before the sequential signals are processed, a final image must be reconstructed from the preliminary image. An alignment technology refers to a paper, which is written by B. G. Zager, R. J. Fomaris, and D K. W. Ferrara, “Ultrasonic Mapping of the Microvasculature: Signal Alignment”, Ultrasound in Med. & Biol., Vol. 24, No. 6, pp. 809-824, 1998. The alignment technology is to calculate the function of cross correlation between any adjacent two of all ultrasonic signals in the same preliminary image. The ultrasonic transducer transmits the ultrasonic pulses when the distance of the horizontal motion of the ultrasonic transducer is 1 μm, and this distance is relatively smaller than the beam (i.e. lateral resolution) of the focal zone of the ultrasound. Thus, the adjacent two ultrasonic signals have high cross correlation. According to the property of high cross correlation, the ultrasonic signal is moved upward or downward to a position having the highest cross correlation with the previous signal so as to finish the reconstruction of the final image, shown in FIG. 9.

The alignment technology is described as follows: pᵢ, pᵣ represent tow ultrasonic signals to be calculated by the cross correlation, pᵢ is any ultrasonic signal on the preliminary image, and pᵣ is standard ultrasonic signal, wherein the function of cross correlation is expressed as

$${\hat{R}}_{{p}_{i} , {p}_{r}} ( m ) = \{ \begin{matrix}\frac{1}{N - m} {\sum }_{n = 0}^{N - m - 1} {p}_{i} ( n + m ) {p}_{r} ( n ) , 0 \leq m < N - 1 \\ \frac{1}{N - \vert m \vert} {\sum }_{n = 0}^{N - \vert m \vert - 1} {p}_{i} ( n ) {p}_{r} ( n + \vert m \vert ) , - ( N - 1 ) \leq m < 0\end{matrix}$$

N is the length of ultrasonic signal. Since the previous ultrasonic signal is set to an alignment standard of any ultrasonic signal on the preliminary image, i=r+1. The maximum value of the function of cross correlation is corresponding to m, i.e. the position having the highest cross correlation between the two ultrasonic signals. Thus, the necessary time shift t by aligning pᵢ is expressed as

$$t = {S}^{t} ( {p}_{i} ) {\vert}_{{p}_{r}} = arg \{ {max}_{m} \{ {\hat{R}}_{{p}_{i} , {p}_{r} ( m )} \} \}$$

The calculation of the flow velocity in the third object on the surface of the first object:

The flow velocity in the third object on the surface of the first object is calculated by 1D-auto correlation function. The 1D-auto correlation function refers to a paper, which is written by C. Kasai, K. Namekawa, A. Koyano, and R. Omoto, “Real-Time Two-Dimensional Blood Flow Imaging Using An Autocorrelation Technique”, IEEE Trans. Sonics Ultrason., vol. SU-32, pp. 458-464, 1985.

The example of the present invention:

The high-frequency ultrasonic imaging system includes an ultrasonic transducer (model V324, GE Panametrics, Waltham, Mass., USA) with a central frequency of 25 MHz and a spatial resolution of 150 μm. The ultrasonic transducer is mounted on the servo-motor for driving the ultrasonic transducer to scan the object. A pulser/receiver (PR5900, Panametrics, USA) utilizes the ultrasonic transducer to transmit high-frequency ultrasonic pulses and receive echoes. The received echoes (RF signals) are amplified by an amplifier (model 150A100B, AR, USA), are transferred by an analogy-to-digital converter (PCI-9820, AdLink INC, Taiwan) to digital data analyzed by the personal computer, and then stored in the memory of the personal computer. Finally, the program of the personal computer displays the image and analyzes the digital data.

In order to prove that the skin scanning method is better than the swept scanning method, a phantom experiment is designed so as to verify that the skin scanning method improves the accuracy of flow estimation. An object phantom having a curved surface is simulated to a tumor having an irregular surface. The object phantom is constituted by water of 100 g, gelatine powder of 2 g and graphite powder of 1.5 g. Three plastic tubes are simulated to blood vessels and put in the object phantom, and the diameters of the three plastic tubes are 1 mm. The Doppler angles of the three plastic tubes are 46.57 degrees. After the object phantom is finished, the blood phantom (model 046, CIRS, VA, USA) is injected by syringe pump (KDS100, KD Scientific, MA, US) so as to simulate the scattering property of the erythrocyte. The flow velocities of the blood phantom in the plastic three tubes are set to 40 ml/hr. After the flows are steady, the images of the flow phantom can be acquired by the skin scanning method and the swept scanning method, respectively.

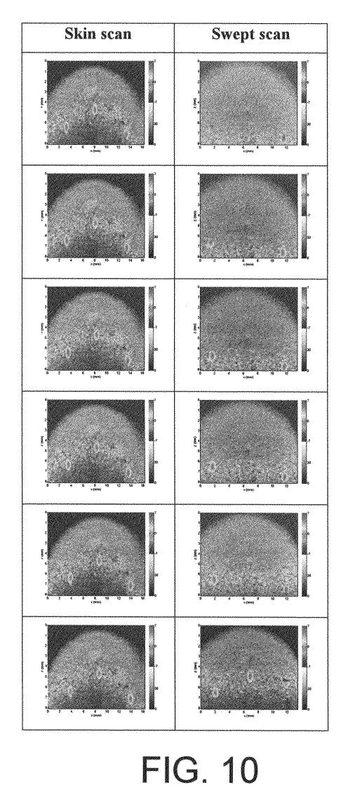

FIG. 10 is a color Doppler image of a flow phantom showing the scanning result of the three blood vessels during the skin scanning method and the swept scanning method. The shape of the three blood vessels is not deformed during the skin scanning method, but the shape of some blood vessels in the non-focal zone is seriously deformed during the swept scanning method. For more serious condition, the shape of some blood vessel displayed by the swept scanning method can be ignored, because some blood vessel are viewed as noise when the distribution of flow velocity is superimposed to B-mode image. More detailed, when a color Doppler image of a flow phantom is made and the distribution of flow velocity is superimposed to B-mode image, there is an importance basis for judgement. Since the reflection ability of the erythrocyte to ultrasonic signals is weaker than that of the tissue to ultrasonic signals, the intensity of flow velocity in the blood vessel must be less that of the tissue and more than the noise so as to avoid ignoring the flow velocity in the blood vessel. However, the signal/noise ratio (SNR) of the non-focal zone is evidently worse than that of the focal zone. If the erythrocyte with weaker reflection ability is located in the non-focal zone, the intensity level of flow velocity in the blood vessel is nearly decreased to that of the noise. Thus, some blood vessel will be viewed as noise when the distribution of flow velocity is superimposed to B-mode image.

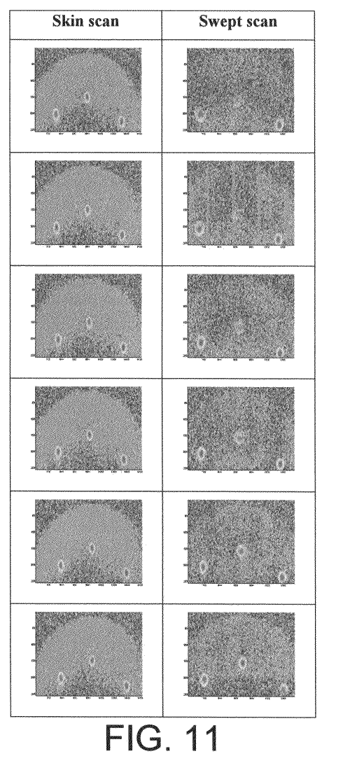

FIG. 11 shows the distribution of the blood flow velocities in the three blood vessels during the skin scanning method and the swept scanning method. The blood flow estimation in the three blood vessels is precise during the skin scanning method, but the blood flow estimation in the three blood vessels in the non-focal zone is un-precise during the swept scanning method. More detailed, when the blood phantom flows through the blood vessels, a portion of the blood phantom which contacts a wall of the blood vessels can be affected by a viscous force. Thus, the outer portion of the blood phantom which contacts the wall of the blood vessel has slower flow velocity, and the inner portion of the blood phantom which is near a center of the blood vessel has faster flow velocity. The flow of the blood phantom which is near the center of the blood vessel is similar to a laminar flow. The above-mentioned property of the simulated blood vessel can be displayed during the skin scanning method, but the distribution of the flow velocity in the simulated blood vessel in the non-focal zone shows turbulent flow during the swept scanning method.

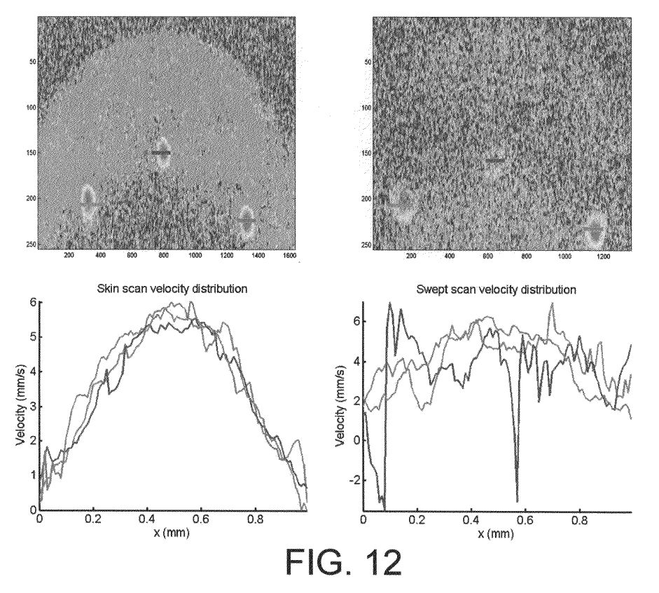

FIG. 12 shows that the distribution of the blood flow velocities in the cross-sections of the three blood vessels during the skin scanning method and the swept scanning method. The distribution of the blood flow velocity in the cross-section of each blood vessel is normal during the skin scanning method, but the distribution of the blood flow velocity in the cross-section of each blood vessel in the non-focal zone is abnormal during the swept scanning method. More detailed, the red line, the blue line and green line indicate left, upper and right simulated blood vessels respectively, and FIG. 12 shows the flow estimation in the cross-section of each blood vessel in the non-focal zone is extremely unstable during the swept scanning method.

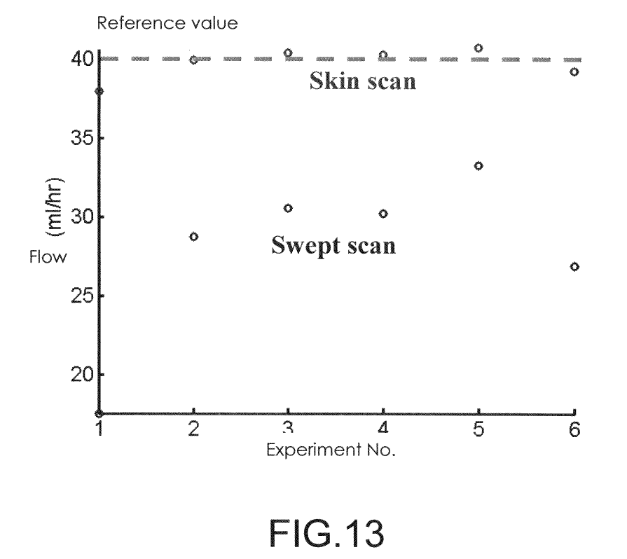

FIG. 13 shows the flow estimation in the three blood vessels in six experiments during the skin scanning method and the swept scanning method. According to formula of Flow, flow is equal to flow velocity multiplied by cross-section area, i.e. Q=VA. However, the shape of each blood vessel is deformed, and the flow estimation in the cross-section of each blood vessel in the non-focal zone is extremely un-precise during the swept scanning method. Compared with the reference value 40 ml/hr, i.e. the flow velocities of the blood phantom in the three blood vessels is injected by syringe pump, the percentage error of the flow estimation in the three blood vessels in six experiments in the skin scanning method and the swept scanning method can be calculated. For the six experiments, the percentage error of the flow estimation of the swept scanning method is evidently higher than that of the skin scanning method. The percentage error of the flow estimation in some experiments in the swept scanning method is near −30%, and especially the percentage error of the flow estimation of the non-focal zone in some experiments in the swept scanning method is above −50%. However, the percentage error of the flow estimation in six experiments in the skin scanning method can be within ±5%.

In conclusion, for a single element high-frequency ultrasonic transducer, the shape of some blood vessels in the non-focal zone is seriously deformed, and the distribution of the blood flow in the section of each blood vessel in the non-focal zone is abnormal during the swept scanning method. The non-focal zone has the lower spatial resolution and the lower ratio of signal to noise. It is interesting that the growth of tumor depends on that of angiogenesis located on the tumor. Although the angiogenesis grow up on the surface of the tumor which is irregular, the focal zone still covers the whole angiogenesis during the skin scanning method, because the focal zone of the ultrasonic transducer is extended along the contour of the tumor. Thus, the ultrasonic transducer of the present invention which has the specific focal depth can effectively estimate the micro-circulation flow of the angiogenesis located on the surface of the tumor.

The skin scanning method of the present invention can resolve the problem of incorrect estimation of blood flow when the blood flow is measured at the non-focal zone of the high-frequency ultrasound. The present invention utilizes a driving device (e.g. piezoelectricity-ceramic motor) to drive high-frequency ultrasonic transducer. During the scanning steps of a skin scanning method, the present invent dynamically keeps the focal zone of the high-frequency ultrasound along the irregular contour of the first object. Thus, the skin scanning method of the present invention can be applied to the other fields as follows: a blood flow measurement of the cortex in the brain, a diagnosis of the surface on the burned skin, and a recovery estimation of blood flow arrangement.

It will be readily seen by one of ordinary skill in the art that one or more embodiments according to the present invention fulfill one or more of the objects set forth above. After reading the foregoing specification, one of ordinary skill will be able to affect various changes, substitutions of equivalents and various other embodiments of the invention as broadly disclosed herein. It is therefore intended that the protection granted hereon be limited only by the definition contained in the appended claims and equivalents thereof.

What is claimed is:

1. An ultrasonic imaging system for estimating a surface of a first object located in a second object, the ultrasonic imaging system comprising:
an ultrasonic transducer for transmitting high-frequency ultrasonic pulses and receiving echoes;
a driving device physically connected to the ultrasonic transducer for driving the ultrasonic transducer to move along a scanning route parallel to a contour of the first object; and
an electronic device electrically connected to the driving device and the ultrasonic transducer for controlling the driving device, acquiring the contour of the first object, processing the received echoes, reconstructing an image and displaying the image.

2. The system of claim 1, wherein the driving device drives the ultrasonic transducer to simultaneously move horizontally and vertically.

3. The system of claim 1, wherein the ultrasonic transducer has a specific focal depth for forming a focal zone extended along the contour of the first object.

4. The system of claim 1, wherein the first object is a tumor, and the second object is one of a human body and an animal body.

5. The system of claim 4, wherein a third object is located on the surface of the first object.

6. The system of claim 5, wherein the third object is angiogenesis.

7. The system of claim 6, wherein the electronic device is adapted for calculating a flow velocity in the angiogenesis.

8. The system of claim 1, wherein the driving device is a servo-motor mechanism.

9. An ultrasonic imaging method for estimating a surface of a first object located in a second object, the ultrasonic imaging method comprising the following steps of:
acquiring a contour of the first object;
moving an ultrasonic transducer along a scanning route parallel to the contour of the first object for transmitting high-frequency ultrasonic pulses and receiving echoes;
forming a focal zone by a specific focal depth of the ultrasonic transducer during moving, wherein the focal zone is extended along the contour of the first object for estimating the surface of the first object;
processing the received echoes resulted from the focal zone so as to form a preliminary image;
reconstructing the preliminary image to a final image from; and
displaying the final image.

10. The method of claim 9, wherein the step of acquiring a contour of the first object comprising the following steps of:
executing a prior scan for the first object by brightness scanning steps; and
calculating the contour of the object by a program.

11. The method of claim 9, wherein the step of reconstructing the preliminary image to a final image comprising the following step of:
calculating a function of cross correlation between any adjacent two of all ultrasonic signals in the same preliminary image.

12. The method of claim 9, wherein the driving device drives the ultrasonic transducer to simultaneously move horizontally and vertically.

13. The method of claim 9, wherein the ultrasonic transducer has a specific focal depth for forming a focal zone extended along the contour of the first object.

14. The method of claim 9, wherein the first object is a tumor, and the second object is one of a human body and an animal body.

15. The method of claim 14, further comprising the following step of:
calculating a flow velocity in a third object on the surface of the first object.

16. The method of claim 15, wherein the third object is angiogenesis, and the flow velocity is a blood flow velocity.

17. The method of claim 15, wherein the flow in the third object on the surface of the first object is calculated by 1D-auto correlation function.

18. The method of claim 9, wherein the method is applied to one of fields of a blood flow measurement of the cortex in the brain, a diagnosis of the surface on the burned skin, and a recovery estimation of blood flow arrangement.
